(19) United States
(12) Patent Application Publication
(10) Pub. No.: US 2017/0165299 A1
(43) Pub. Date: Jun. 15, 2017

(54) COMPOSITIONS AND METHODS RELATING TO TREATMENTS OF THE ORAL CAVITY

(71) Applicant: REPLICEL LIFE SCIENCES INC., Vancouver (CA)

(72) Inventors: Rolf Hoffmann, Freiburg (DE); Kevin John McElwee, Vancouver (CA)

(73) Assignee: REPLICEL LIFE SCIENCES INC., Vancouver, BC (CA)

(21) Appl. No.: 15/118,383

(22) PCT Filed: Feb. 12, 2015
(86) PCT No.: PCT/US15/15720

§ 371 Date: Aug. 11, 2016

Related U.S. Application Data
(60) Provisional application No. 61/939,157, filed on Feb. 12, 2014.

(51) Int. Cl. A61K 35/36; A61L 27/36; A61L 27/54; A61L 27/58; C12N 5/071; A61L 27/38

(52) U.S. Cl. CPC A61K 35/36; C12N 5/0627; A61L 27/3804; A61L 27/54; A61L 27/58; A61L 27/3633

(57) ABSTRACT

The present invention relates to compositions and methods utilizing hair follicle derived Non-Bulbar Dermal Sheath cells for use in the treatment of dental-related conditions, including the treatment of gingivitis. Briefly stated, the present invention provides compositions and methods for treating or preventing a wide variety of conditions associated with the oral cavity, including for example, periodontal disease and a variety of diseases or injuries associated with the gingiva utilizing hair follicle derived Non-Bulbar Dermal Sheath (“NDBS”) cells. Within one aspect of the invention methods are provided for isolating NBDS cells, comprising the steps of: preparing vital hair and cleaving the hair.

### CROSS-REFERENCE TO RELATED APPLICATION

This application claims the benefit under 35 U.S.C. §119(e) of U.S. Provisional Patent Application No. 61/939,157 filed Feb. 12, 2014, which application is incorporated herein by reference in its entirety.

### FIELD OF THE INVENTION

The present invention relates to compositions and methods for treating the oral cavity, and more specifically, to compositions comprising autologous or allogeneic non-bulbar dermal sheath (NDBS) cells for use in a wide variety of treatments of an oral cavity, including for example, the treatment and repair of gingiva.

### BACKGROUND

### Description of the Related Art

Disease, injuries and trauma associated with the oral cavity (i.e., the mouth and associated structures) are one of the most prevalent chronic diseases worldwide, and a costly burden to health care. The treatment of diseases associated with the oral cavity accounts for between 5% and 10% of total healthcare expenses in industrialized countries. In most developing low-income countries, the prevalence of dental caries alone is more than 90%.

One of the most common dental diseases are gingival recessions (receding gums), which are characterized by a loss of gum tissue and/or retraction of the margins of the gingiva from the crown of the teeth. Receding gums are a common problem in adults over the age of 40, but it may also occur starting even in teenagers. It may exist with or without a concomitant decrease in the crown-to-root ratio which is due to recession of alveolar bone.

As shown in FIG. 1, FIG. 1A illustrates a normal tooth, and FIG. 1B a tooth with shrinked gingiva, tooth rot and the building of a gingival pocket. These pockets are then ideal places for bacterial growth, which in turn leads to gingival inflammation and destruction of alveolar bone and eventually loss of teeth.

[symbol]

condition that occurs gradually over many years. Therefore it is common over the age of 40. The changes in gum volume do not change much from one day to another and therefore the progression is minimal and patients get used to the gums' appearance and tend not to notice the recession visually. In fact, receding gums may remain largely unnoticed until the condition starts to cause symptoms.

Therefore, there is a need in the art for prophylactic or early treatment, before symptoms begin. While there are a number of surgical and non-surgical methods that can be used to treat the gingiva, none of those techniques addresses the issue of a cellular deficit of functional fibroblasts in the aged, injured gingiva.

The present invention discloses novel compositions and methods for treatments of the oral cavity, such as those involving the gingiva and oral mucosa, and further provides other related advantages.

The details of one or more embodiments are set forth in the description below. Other features, objects and advantages will be apparent from the description, the drawings, and the claims. In addition, the disclosures of all patents and patent applications referenced herein are incorporated by reference in their entirety.

### SUMMARY

Briefly stated, the present invention provides compositions and methods for treating or preventing a wide variety of conditions associated with the oral cavity, including for example, periodontal disease and a variety of diseases or injuries associated with the gingiva utilizing hair follicle derived Non-Bulbar Dermal Sheath (“NDBS”) cells. Within one aspect of the invention methods are provided for isolating NBDS cells, comprising the steps of: (a) preparing vital hair; (b) cleaving the hair prepared in step (a) to remove the hair follicle bulb (which contains the dermal sheath cup and dermal papilla); (c) isolating Non-Bulbar Dermal Sheath tissue; and (d) cultivating the isolated Non-Bulbar Dermal sheath tissue to produce NBDS cells. The use of NBDS cells can either be autologous or allogeneic. Within one embodiment of the invention the vital hair is obtained by full skin biopsy from the occipital scalp of a subject. Within another embodiment the hair is cleaved utilizing a micromanipulator such as a needle along with a scalpel or pair of scissors. Within yet other embodiments, digestion of the isolated Non-Bulbar Dermal Sheath tissue, optionally, with, for example collagen digesting enzymes such as collagenase, hyaluronidase, DNAse, elastase, papain, protease type XIV, trypsin, dispase, and leupeptin. Within further embodiments, the cells are passaged over multiple passages.

Within other aspects of the invention, isolated NBDS cells are provided, optionally prepared according to the methods described above. These NBDS cells may be contained within compositions with other ingredients, such as, for example, blood plasma, blood serum, platelet-rich plasma (PRP), fibrin, and/or hyaluronic acid. Other ingredients may also be included within these compositions, including for example, components of the extracellular matrix (e.g., glycosaminoglycans (GAGs), heparin sulfate, chondroitin sulfate, keratin sulfate, hyaluronic acid, elastin, fibronectins, fibrin, composite scaffolds, collagens and laminins, or any other soluble or insoluble extracellular matrix), cytokines and chemokines (e.g., transforming growth factor beta (TGF-beta) and its isoforms, insulin-like growth factor (IGF) and its isoforms, granulocyte-macrophage colony-stimulating factor (GM-CSF), parathyroid-hormone-related protein, hepatocyte growth factor/scatter factor (HGF/SF), macrophage stimulating protein (MSP), epidermal growth factor (EGF), interleukin 6 (IL-6), stromal cell-derived factor 1 (SDF-1), platelet derived growth factor (PDGF) and fibroblast growth factor (FGF) and/or various therapeutic agents (e.g., analgesic agents, anti-inflammatory agents antibiotics, antimycotics and immunomodulatory agents).

Within yet other aspects of the invention methods are provided for treating the gingiva of a subject, comprising the step of administering to the gingiva of a subject a composition comprising NBDS cells as described herein. Within one embodiment, the subject is a mammal selected from the group consisting of humans, horses, dogs and cats. Within various embodiments the treatment is due to a gingival injury. Within certain embodiments, the gingiva injury can result from external trauma (e.g., a surgical procedure or wound, piercings, burns, radiation, or an accident), or an acute or chronic wound or scar. Within other embodiments the gingiva injury is because of a predisposition of spontaneous or induced fragile mucosa such as such as epidermolysis bullosa aquisita, autoimmune diseases with blisters, wounding or atrophy such as due to pemphigus and others. Within other embodiments gingiva can age or be injured, with abnormal tooth position, such as the crowding of teeth which gives rise to inadequate cover of one or more teeth by the jaw bone, inherited insufficient gingival tissues, acquired diseases with fragility, blistering, insufficient healing, chronic wounding such as epidermolysis bullosa aquisita, autoimmune diseases with blisters, wounding or atrophy such as due to pemphigus and others, overaggressive brushing, periodontal diseases, improper flossing or brushing which allows bacteria to build up between the teeth and at plaques, self-induced vomiting or other eating disorders, shrinkage of gingival tissue due to ageing or by the use of chewing tobacco or the adverse effects of smoking, teeth grinding, scurvy and other nutritional deficiencies which affect proper growth of gums, or sensitivity to detergents. In other embodiments gingival recessions may also be caused by gingivitis which then is associated with puffy red, swollen gums, bleeding and bad breath.

Within yet other embodiments gingival injuries may incur by acute or chronic viral, mycotic or bacterial infections, chronic autoimmune inflammatory disease such as Scleroderma and variants, Borelliosis infection, Lupus erythematosis and variants, Lichen ruber planus and general aging (intrinsic aging). Within yet another embodiment, the gingiva is on the entire oral mucosa, including the soft and hard palate, or a selected portion of the jaws such as individually affected teeth. Within another embodiment the treatment is meant to be prophylactic in order to avoid irreversible gingiva shrinkage.

### BRIEF DESCRIPTION OF THE DRAWINGS

FIGS. 1A and 1B illustrate a normal tooth (1A), and a tooth (1B) with shrinked gingiva, tooth rot and the building of a gingival pocket.

FIG. 2 illustrates the dissection of a human hair follicle. FIG. 2A shows an isolated human hair follicle, which can be cleaved above the bulbar portion of the hair root (i.e., above the dermal papilla and dermal sheath cup cells, i.e., above the end bulbs), but below the base of the sebaceous gland canal, in order to obtain an isolated dermal sheath (see FIG. 2B). The structure depicted in FIG. 2B can be separated into at least two separate components, as shown in FIGS. 2C and 2D. FIG. 2C depicts the hair fiber and associated inner root sheath, and outer root sheath which NBDS cells (also occasionally referred to as the connective-tissue sheath, upper dermal sheath or less precisely just dermal sheath). In contrast to many other types of cells, NBDS cells are highly positive for a collagen-1 marker and only weakly positive for alkaline phosphatase and steroid sulfatase. In addition, these cells express markers such as CD90 and other stem cell markers.

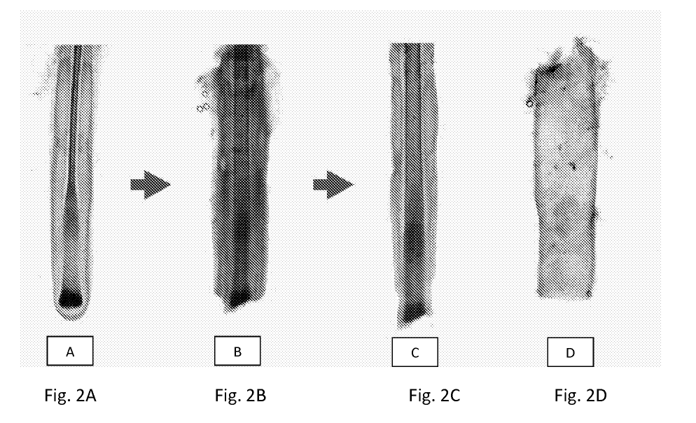

FIG. 3 is a photomicrograph of NBDS cells in culture.

### DETAILED DESCRIPTION OF THE INVENTION

As noted above, the present invention provides hair follicle derived Non-Bulbar Dermal Sheath (NDBS) cells for use in the treatment or prevention of a wide variety of dental conditions, diseases and or trauma (e.g., surgical wounds), including for example, treatment of the gingiva. Prior to setting forth the invention however, it may be helpful to an understanding thereof to first set forth definitions of certain terms that are used hereinafter.

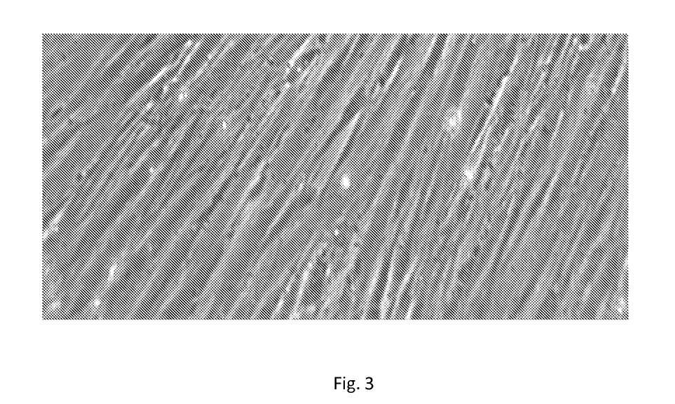

Non-Bulbar Dermal Sheath cells, or “NBDS” cells, refers to dermally derived cells (or more specifically, derived from hair follicles). Within preferred embodiments, the sheath cells are obtained from the outer dermal sheath of a hair follicle, above the bulbar portion of the hair root (i.e., above the dermal papilla and dermal sheath cup cells), but below the base of the sebaceous gland canal. NBDS cells may be readily identified by a number of methods, including for example, by the method of preparation and culture (as described below); morphology (see, e.g., FIG. 3); as well as cell specific markers (e.g., NBDS cells are primarily positive for CD 90, CD73 and CD49b, and/or primarily negative for CD34, CD45 and KRT14, either before or after culturing). In all events however, the cells must be of a dermal origin, and more specifically, of a follicular origin.

Expanded Non-Bulbar Dermal Sheath cells, or “eNBDS cells” refers to NBDS cells which have been expanded for several passages in culture, but which retain the ability to produce collagen (e.g., type I collagen) as well as a variety of cytokines and chemokines. As above, unexpectedly, the eNBDS cells can also be immunoregulatory. Within preferred embodiments, the cells can be expanded in culture for 1, 2, 3, 4, 5, 10, 20 or more passages.

[symbol]

90%, 95%, 98% or 100% NBDS cells. NBDS cells have the ability to produce collagen (e.g., type I collagen), as well as a variety of cytokines and chemokines. Unexpectedly, the NBDS cells can also be immunoregulatory, making them particularly suitable for treatment of tendon injuries (e.g., by assisting in suppressing any inflammatory response).

Within certain embodiments of the invention, software or other visualization techniques that can be utilized to visualize cells on a microscopic scale can be used to assess the size, shape, viability and granularity of a large number of cells in a visual field, and ascertain the number of NBDS cells (which are fibroblast-like—as shown in FIG. 3), as opposed to keratinocytes, melanocytes DSCs, and other cell types which are of different morphology). Hence, within one embodiment of the invention methods are provided for isolating NBDS cells comprising the step of culturing cells over at least 1, 2, 3, 4, 5, 6, 10, or 20 passages from a hair follicle such that an isolated population of NBDS cells is produced. Within preferred embodiments the cells placed into dishes or flasks which allow the NBDS cells to adhere, and with each passage non-adherent cells are removed, and the remaining adherent cells released (e.g., by trypsinization), followed by addition of fresh media. Within such embodiments it can be determined when a sufficient population of isolated NBDS cells has been obtained by visualizing the cells in the cell culture in order to assess the number of NBDS cells vs. non-NBDS cells. Visualization techniques include, but are not limited to direct microscopic visualization, staining of the cells for markers (or lack thereof—e.g., for lack of keratin), and light/laser analysis to look at diffraction patterns of the different cell types (see, generally “Laser Scanning Microscopy and Quantitative Image Analysis of Neuronal Tissue” Lidia Bakota and Roland Brandt, eds., Humana Press, 2014; see also “Imaging and Spectroscopic Analysis of Living Cells: Optical and Spectroscopic Techniques”, Conn ed., Academic Press, 2012)

Within other embodiments, cell specific markers (e.g., NBDS cells are primarily positive for CD 90, CD73 and CD49b, and/or primarily negative for CD34, CD45 and KRT14 (optionally before or after culturing) can be utilized to assess the degree of NBDS cells vs. contaminant cell types. (“Applications of Flow Cytometry in Stem Cell Research and Tissue Regeneration”, Krishan, Krishnamurthy, and Totey eds., obtaining one or more vital hair follicles; b) releasing cells from the hair follicle (e.g., through the use of enzymes, or by culturing growing cells out of the hair follicle); and c) sorting the cells (e.g., by flow cytometry or through the use of magnetic beads) to obtain a population of isolated NBDS cells. Within certain embodiments of the invention cells in any stage of the process may be optionally cultured as described above (e.g., cells may be cultured for at least 1, 2, 3, 4, 5, 6, 10 or 20 passages as described above, and the resultant cells further isolated by, for example, flow cytometry or magnetic beads.

Within preferred embodiments the isolated NBDS cells are at least 70%, 80%, 90%, 95%, 98% or 100% positive for one or more of the positive markers described above, and/or at least 80%, 90%, 95%, or 98% negative for one of the negative markers described above.

Within preferred embodiments of the invention (and utilizing any of the techniques described herein), isolated NBDS cells have less than 15%, 10%, 5%, or 1% keratinocytes within the cell population and/or less than 15%, 10%, 5%, or 1% melanocytes within the cell population. However, within further embodiments, the isolated NBDS cell population is derived from a population of dermal cells (preferably, from hair follicles) that have some contaminating cell types, including for example, at least 5, 10, 0.01%, 0.1%, or 1% keratinocytes in the cell population, and/or at least 5, 10, 0.01%, 0.1%, or 1% melanocytes. Within further embodiments of the invention the isolated NBDS cells are at least 95% pure, and have at least one contaminating cell type (e.g., at least one keratinocyte) within the cell population.

“Gingival injury” refers to the loss or shrinkage of gingiva due to, for example, external trauma (e.g., a surgical procedure or wound, piercings, burns, radiation, or an accident), or an acute or chronic wound or scar. A predisposition of spontaneous or induced fragile mucosa such as such as epidermolysis bullosa aquisita, autoimmune diseases with blisters, wounds or atrophy such as pemphigus and others. The gingiva can age or be injured, leading to receding gums and potentially alveolar bone destruction because of abnormal tooth position, such as the crowding of teeth which gives rise to inadequate cover of one or more teeth by the jaw bone, inherited insufficient gingival tissues, acquired diseases with fragility, blistering, insufficient with blisters, wounding or atrophy such as pemphigus and others, overaggressive brushing, periodontal diseases, improper flossing or brushing which allows bacteria to build up between the teeth and at plaques, self-induced vomiting or other eating disorders, shrinkage of gingival tissue due to ageing or by the use of chewing tobacco or the adverse effects of smoking, teeth grinding, scurvy and other nutritional deficiencies which affect proper growth of gums or sensitivity to detergents. Gingival recessions may also be caused by gingivitis which then is associated with puffy red, swollen gums, bleeding and bad breath. In addition gingival injuries may incur by acute or chronic viral, mycotic or bacterial infections, chronic autoimmune inflammatory disease such as Scleroderma and variants, Borelliosis infection, Lupus erythematosis and variants, Lichen ruber planus and general aging (intrinsic aging). Within yet another embodiment, the gingiva is on the entire oral mucosa, including the soft and hard palate, or a selected portion of the jaws such as individually affected teeth.

### Preparation of NBDS

As noted above, the present invention provides methods for isolating NBDS cells. Within one aspect of the present invention such methods comprise the steps of (a) preparing vital hair; and (b) culturing the vital hair such that a population of NBDS cells can be obtained. With respect to step (a), a wide variety of methods may be utilized to obtain vital hair, including for example, surgical methods to remove a variety of hair follicles (along with the skin), or by plucking one or more hair follicles directly from a subject.

Once the vital hair has been obtained, it can be cultured under conditions which allow, and preferentially, promote the growth of NBDS cells. Within preferred embodiments, this culturing under conditions wherein fibroblast-like cells are allowed to proliferate. Within preferred embodiments the step of culturing is performed with serum-free media. After several passages (e.g., at least 1, 2, 3, 4, 5, 6, 10 or 20 or more passages), the cultured cells are analysed as described above in order to ascertain whether there is a sufficient quantity of NBDS cells, and whether the cells have been sufficiently isolated from contaminating cells.

Within other aspects of the invention, methods are provided comprising the hair follicle bulb (which contains the dermal sheath cup and dermal papilla); (c) isolating Non-Bulbar Dermal Sheath tissue; and (d) cultivating the isolated Non-Bulbar Dermal sheath tissue to produce NBDS cells.

In order to prepare vital (or ‘living’) hair, a sample is typically obtained from a given subject (e.g., a mammal such as a human, horses, pigs, cats, dogs, rabbits, guinea pigs, rats or mice). The sample may be obtained from a variety of sites (e.g., for humans, from the occipital area of the scalp, the chest or thigh, and for horses from the mane or tail). The sample may be obtained via a biopsy, or other suitable means (e.g., by ‘plucking’, or dissection). Preferably, hair follicles in the anagen phase of development are selected, although other phases of development (e.g., the catagen phase or telogen phase) can also be utilized.

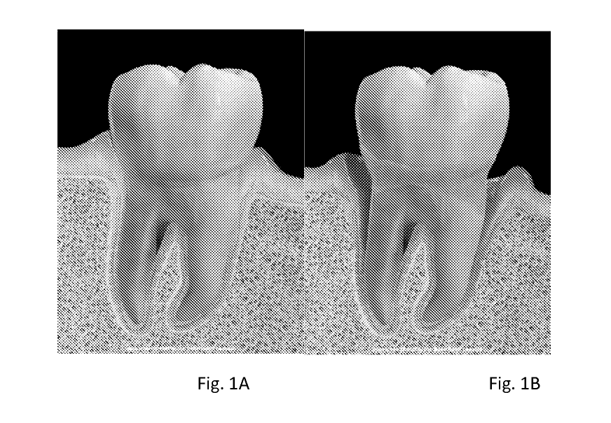

Once the sample is obtained from the subject, the sample is then separated to isolate the hair follicle, typically utilizing a micromanipulator and scalpel, although other instruments such as needles may also be utilized. Within certain embodiments, the isolated hair follicle as shown in FIG. 1A can be further cleaved above the bulbar portion of the hair root (i.e., above the dermal papilla and dermal sheath cup cells), but below the base of the sebaceous gland canal in order to obtain an isolated dermal sheath (see FIG. 1B). The structure depicted in FIG. 1B can be separated into at least two separate components, as shown in FIGS. 1C and 1D. FIG. 1C depicts the hair fiber and associated inner root sheath, and outer root sheath which contain predominantly keratinocytes, and FIG. 1D is the dermal sheath containing NBDS cells (also occasionally referred to as the connective-tissue sheath).

The dermal sheath (FIG. 1D) can, within certain embodiments, be further separated, for example, by cutting length-wise along one side, or, by using techniques such as enzymatic digestion (e.g., with collagen digesting enzymes such as collagenase, dispase and leupeptin).

The dermal sheath containing NBDS cells, or the separated NBDS cells can then be cultured in a medium (either with or without serum) which promotes cell proliferation (see e.g., FIG. 3). Suitable media include, for example, DMEM/Hams F12 supplemented with fibroblast growth factor (FGF), fetal calf/bovine serum and antibiotics. Alternatively, cells can be replicated in a serum-free process, in which of serum-free media include X-Vivo™ and TheraPEAK™ FGM-CD™ containing serum supplements and/or human derived platelet extract. After 3 to 5 days, fresh proliferation medium is typically added to the culture medium. Subsequently the medium can be changed every 2 to 4 days. When the culture has reached approximately 80 to 90% confluence, the cells are detached from the culture flask via trypsinization and seeded in a larger tissue culture flask. This step is repeated for a number of passages (e.g., 2, 4, or 6) until approximately 5 to 100 million cells are obtained.

Once the desired number of cells are obtained, the cells are washed several times, trypsinized, and resuspended in cell transportation medium (CTM), which is composed of ringer lactate, 10% human serum albumin (HSA) and 5% dimethylsulfoxide (DMSO). Cells are counted and adjusted to provide the final concentration of 20 million cells/ml and stored in liquid nitrogen, or optionally stored in suitable media.

Within certain embodiments of the invention cell culture supernatants need not be discarded as they contain individual growth factors, matrix molecules, and stem cell factors, made by the patient's cells. Cell culture supernatants can be frozen, freeze-dried or any other storage method to be suitable for the specific use.

### Preparation of Compositions Comprising NBDS Cells

As noted above, NBDS cells (including isolated NBDS cells) may be contained within compositions with a variety of ingredients, such as, for example, blood serum or plasma, albumin (e.g., human), platelet-rich plasma (PRP), fibrin, and/or hyaluronic acid. Other commercially available products may also be utilized to prepare suitable compositions, including for example, TISSEEL and COSEAL (available from Baxter), TISSUCOL, BERIPLAST, QUIXIL, TACHOSIL, and EVICEL. Other polymer-based compositions may also be utilized, including for example, polyethylene glycols, poly-lactic acids, and poly caprolactones. Within other embodiments, the cells may be placed in either manufactured or harvested extracellular matrices (e.g. US 2010/0047305 or US 2010/0124573, both of which are incorporated by reference in their entirety). Within other embodiments, the cells may be placed within a non-as GINTUIT by Organogenesis, See: http://www.organogenesis.com/products/oral-regeneration.html. Particularly preferred scaffolds or structures include biodegradable scaffolds (e.g., collagen-based scaffolds, such as, for example, meshes, or scaffold combining other cell types e.g. keratinocytes). Representative examples of suitable scaffolds include, for example, U.S. Pat. Nos. 5,736,372, 5,759,830, 8,039,258 and 8,105,380, and U.S. Publication Nos. US 2002/0172705, US 2009/0130068, US 2009/142836, and US 2011/0293667, all of which are incorporated by reference in their entirety.

Within other preferred embodiments the composition is provided in combination or as an adjunct to Guided Tissue Regeneration (or “GTR”). Briefly, GTR is a surgical procedure that specifically aims to regenerate the periodontal tissues. Guided tissue regeneration or GTR is a surgical procedure that utilizes barrier membranes to direct the growth of new bone and gingival tissue at sites having insufficient volumes or dimensions of bone or gingiva for proper function, esthetics or prosthetic restoration.

Within other preferred embodiments the composition is provided in one or two or more parts (e.g., in a double barrelled syringe that admixes components, or in bi- or multichambered cartridge) that is freely flowing and injectable. Representative examples of such syringes include those described in U.S. Pat. Nos. 5,750,657 and 8,039,021, which are both incorporated by reference in their entirety.

Other ingredients may also be included within these compositions, including for example, components of the extracellular matrix (e.g., glycosaminoglycans (GAGs), heparin sulfate, chondroitin sulfate, keratin sulfate, hyaluronic acid, elastin, collagens, fibronectins and laminins), cytokines and chemokines (e.g., transforming growth factor beta (TGF-beta) and its isoforms, insulin-like growth factor (IGF) and its isoforms, granulocyte-macrophage colony-stimulating factor (GM-CSF), parathyroid-hormone-related protein, hepatocyte growth factor/scatter factor (HGF/SF), macrophage stimulating protein (MSP), epidermal growth factor (EGF), interleukin 6 (IL-6), stem cell factor (SCF) stromal cell-derived factor 1 (SDF-1), platelet derived growth factor (PDGF) and fibroblast growth factor (FGF) and/or various therapeutic agents (e.g., analgesic agents, anti-inflammatory agents, antibiotics, antimycotics, antiviral and

### Treatments of the Oral Cavity Utilizing NBDS Cells

Compositions and methods are also provided for treating or preventing a variety of conditions relating to the oral cavity (e.g., the gingiva), comprising the step of administering to a subject a composition comprising NBDS cells (or isolated NBDS cells) as described above. Typically, cells are administered by injection, although within various embodiments, to the extent a surgical method is employed the cells may be provided directly into, beside or underneath an open wound, gingival pockets, and/or around surgical wounds, teeth, or root canals.

### A. Treatment of the Gingiva Utilizing NDBS Cells

Gingival recessions (receding gums) are defined by the exposure of the roots of the teeth. This is caused by a loss of gum tissue and/or retraction of the margins of the gingiva from the crown of the teeth. Receding gums are a common problem in adults over the age of 40, but it may also occur even in teenagers. It may exist with or without a concomitant decrease in the crown-to-root ratio which is due to recession of alveolar bone.

Hence, within various embodiments of the invention a wide variety of conditions related to the gingiva may be treated utilizing NBDS cells (or isolated NBDS cells). Representative examples of such conditions include, for example: 1) abnormal position of the teeth or a tooth, such as the crowding of teeth which gives rise to inadequate cover of one or more teeth by the jaw bone; 2) inherited diseases, such as primary thin or fragile gingival tissue, insufficient gingival tissues, or inherited diseases with fragility, blistering, insufficient healing, chronic wounding such as epidermolysis bullosa; 3) acquired diseases with fragility, blistering, insufficient healing, chronic wounding such as epidermolysis bullosa aquisita, autoimmune diseases with blisters, wounding or atrophy such as pemphigus; 4) mechanical stresses such as, for example, a) overaggressive brushing and/or improper flossing which allows bacteria to build up between the teeth and at plaques, b) teeth grinding, including for example TMJ disease, and c) mechanical injuries of the guns due to surgical trauma, accidental wounds or injuries, and/or elected injuries such as piercing and tattooing; 5) environmental, adverse effects of smoking, b) sensitivity to detergents, and c) insufficient nutrition due to, for example, self-induced vomiting and other eating disorders, as well as inadequate nutrition due to malabsorption or inadequate dietary intake (e.g., Scurvy); and 6) infection of gum tissues, and associated puffy, swollen, red, and in certain instances, bleeding gums.

### B. Further Procedures Utilizing NBDS Cells or Cell Culture Supernatants

As discussed above, a variety of signs of gingival injuries can be readily treated and/or prevented via intracutaneous (i.c.), intramucosal, submucosal, intradermal or subcutaneous (s.c.), the gingival sulcus, or even inside the ligaments of the teeth holding apparatus (periodontal ligaments, cementum) injections of NDBS cells (or isolated NDBS cells). Within various embodiments of the invention the entire gingiva of a subject (e.g., a human subject) can be treated, although, within but more specific aspects, cosmetic and aesthetic treatments can be provided to the subject. Representative examples of such treatments include, but are not limited to: receding gums at the incisor, canines the premolars or the molars or all teeth.

Within various embodiments, the NBDS cells (or isolated NDBS cells) can be delivered via injections, which can be done with or without local or systemic analgesia or sedation. This can be done with a single or a multi-needle device. In addition, it can be performed either by a single injection as a bolus or multiple, multi-layered injections with different techniques such as criss-cross, feathering or others. The delivered volumes/cell numbers largely depend on the indication and the area to be treated. Typical doses may start from as low as 0.01 ml up to several ml. In certain aspects of the invention the injected cell numbers may range from 10 to billions of cells, and more preferably, from 100, 1,000, 10,000, 100,000, 1,000,000 and/or 10,000,000 up to a billion or more cells. The number of injected cells, will depend on, among other things, the size of the area to be treated, the total number of cells available and the volume injected, as well as the desired degree of efficacy.

In addition, injection of NDBS cells (or isolated NDBS cells) into, around, or underneath an acute or chronic wound will be beneficial for wound healing.

[symbol]

provided for the coating of dental implants to facilitate the ingrowth/healing of dental implants or other materials used in dental surgery.

The following examples illustrate the invention and should not be understood as limiting the scope of the invention.

### Example 1

### Harvest of Cells

A skin biopsy from the occipital area of the scalp is obtained from a subject as follows. Briefly, once an appropriate area of the scalp has been selected, it is shaved with hair clippers, ensuring some stubble remains. The biopsy area is then thoroughly disinfected and anaesthetized. Once anesthesia has taken effect, a 1-10 mm deep punch or an excisional biopsy is gently removed from the biopsy site and the incision closed with sutures which can be removed 8-16 days later. The skin biopsy is then packaged under aseptic conditions into a pre-labelled biopsy tube containing transport medium.

### Example 2

### Isolation and Cultivation of NBDS Cells

A sterility test is performed on the medium in which the biopsy has been transported to ensure the sample is free from contamination, or alternatively, if the sample is contaminated to ensure that medium with antibiotics is subsequently utilized. The biopsy is then washed to remove the biopsy transportation medium and any debris to prepare the tissue for subsequent processing. Hair follicles are removed from skin biopsies by cutting away the skin epithelium with a sterile scalpel and “plucking” or dissecting the whole hair follicle unit from the surrounding dermal tissue using sterile forceps. The hair follicle is gripped with a forceps as close as possible to the skin surface and the follicle exposed by pulling up on the hair in the hair follicle unit. Only, but not restricted to, follicles in the anagen phase (growing phase of the hair cycle, indicated by the visible outer root sheath, and DSC of the hair bulb) are selected for further processing.

The hair follicle bulb including the follicular dermal sheath cup cells and papilla is detached from the rest of the hair follicle using a fine sterile mini-scalpel or mechanically or enzymatically removed, and the tissue is prepared for cultivation.

The cells/tissue are placed in culture with cell proliferation promoting culture medium. After 48-72 hours the proliferation medium is exchanged for fresh medium. Subsequently the medium is changed every 2 to 4 days. When the culture has reached approximately 80%, but not limited to, confluence, the cells are subcultivated. This step is repeated until approximately the desired number of cells is obtained.

Once a sufficient number of cells are obtained (e.g., typically one million or more), the cells are washed with appropriate buffers/media (e.g., PBS, DMEM, Williams E), trypsinized and resuspended in Cell Transportation Medium, such as Hams F10 or DMEM. The cells are sedimented by centrifugation. The supernatant is aspirated and the cell pellet is resuspended and washed again in CTM. The cells are sedimented once more by centrifugation, and the resulting pellet is resuspended in CTM to give a final desired concentration of cells/ml. Cells can be frozen in 1.0 ml ringer lactate supplemented with 10% human serum albumin (HSA) and 5% dimethyl sulfoxide (DMSO).

### Example 3

### Preparation and Administration of NBDS Cells into Gingiva

The gingiva of affected individuals is first prepared for injection by application of a topical analgesia (e.g., spray, cooling) or local injection of a local anesthetic such as Bupivacaine. NBDS cells, prepared as described above, are then injected into the gingiva in a repetitive manner, in order to cover the entire volume of the desired treatment area.

### Example 4

### Use of Cell Culture Supernatants in Various Preparations

As an adjunct, a follow-up or as a standalone treatment, cell culture supernatants can be used to have a beneficial effect on gingival structure, texture, look, moisture, thickness and firmness.

[symbol]

described in Example 2 can be used alone, concentrated or dissolved in typical excipients and applied to aged or injured gingiva.

### Example 5

### Use of Cell Culture Supernatants for Coating Dental/Surgical Implants

Single or multi dental implants are commonly used to treat the absence of teeth. Those implants and other implants used by oral surgeons should integrate into the alveolar bone over time. As the cell culture supernatants of NBDS cells contain growth factors and other factors which might be useful to facilitate healing and proper integration of dental implants into bone. Therefore, the coating of those implants is desirable to reduce fibrous encapsulation and increase biocompatibility of dental implants or other medical devices.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

Fibroblasts are the main cell type in the gingival dermis. They are highly biologically active and for a multi-layered, 3-dimensional network of cells, intermingled with collagen and connected via surface receptors such as integrins. There is a constant renewal and breakdown of collagen fibres by enzymes such as MMP's (matrix metalloproteinases) which regulate the volume and tightness of the skin. With aging or smoking, collagen degrades and cannot be replaced by the aged fibroblasts. The fibroblasts become rather inactive, more round in appearance and produce less collagen. This leads to a more fragile gingiva and this eventually leads to an aged gingiva with receding gums.

The symptoms might be sensitive teeth; A larger part of the teeth is visible and therefore the teeth look longer and unattractive or cosmetically disturbing; even the roots of the tooth are exposed, visible and sensitive; at the gum line the tooth feels between enamel and cementum; the interdental spaces seem to grow and food is going to remain there; cavities may develop below the gum line; eventually loss of alveolar bone or even loss of teeth occurs.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for isolating NBDS cells, comprising:
(a) preparing vital hair;
(b) cleaving the hair prepared in step (a) to remove the hair follicle bulb;
(c) isolating Non-Bulbar Dermal Sheath tissue; and
(d) cultivating the isolated Non-Bulbar Dermal sheath tissue to produce NBDS cells.

2. The method according to claim 1 wherein said vital hair is obtained by biopsy from a hair on the scalp of a subject.

3. The method according to claim 1 wherein isolated NBDS cells can either be used autologous or allogeneic.

4. The method according to claim 1 wherein said hair is cleaved utilizing a micromanipulator and scalpel, or scissors,

5. The method according to claim 1, further comprising the step of conducting enzymatic digestion of said isolated Non-Bulbar Dermal Sheath tissue.

6. The method according to claim 4 wherein said enzymatic digestion is conducted with collagenase hyaluronidase, DNAse, elastase, papain, protease type XIV, trypsin, and dispase.

7. The method according to claim 1 wherein said NBDS cells are passaged over multiple passages.

8. A composition comprising isolated Non-Bulbar Dermal Sheath cells prepared according to the method of any one of claim 1 to 7.

9. A composition comprising isolated Non-Bulbar Dermal Sheath cells.

10. A composition according to claim 8 or 9, further comprising serum, plasma or platelet-rich plasma (PRP)

11. A composition according to any one of claim 8, 9, or 10, further comprising fibrin and/or hyaluronic acid.

12. A composition according to any one of claim 8, 9, or 10, further comprising components of the extracellular matrix, cytokines, chemokines and therapeutic agents. [symbol]
extracellular matrix are selected from the group consisting of glycosaminoglycans (GAGs), heparin sulfate, chondroitin sulfate, keratin sulfate, hyaluronic acid, elastin, collagens, fibronectins and laminins.

14. The composition according to any one of claim 8, 9, or 10, further comprising a scaffold.

15. The composition according to claim 14, wherein said scaffold is a biodegradable scaffold.

16. The composition according to claim 12 wherein said cytokines are selected from the group consisting of transforming growth factor beta (TGF-beta) and its isoforms, insulin-like growth factor (IGF) and its isoforms, granulocyte-macrophage colony-stimulating factor (GM-CSF), parathyroid-hormone-related protein, hepatocyte growth factor/scatter factor (HGF/SF), macrophage stimulating protein (MSP), epidermal growth factor (EGF), interleukin 6 (IL-6), stromal cell-derived factor 1 (SDF-1), platelet derived growth factor (PDGF) and fibroblast growth factor (FGF).

17. The composition according to claim 12 wherein said therapeutic agents are selected from the group consisting of analgesic agents, anti-inflammatory agents antibiotic, antiviral, antimycotic, and immunomodulatory agents.

18. A method for treating an oral cavity, comprising the step of administering to an oral cavity of a subject the composition according to anyone of claims 8 to 17.

19. The method according to claim 1 wherein said step of administering to an oral cavity of a subject comprises the step of administering to the gingiva of a subject.

20. The method according to claim 1 wherein said step of administering to an oral cavity of a subject comprises the step of administering to a dental pocket of a subject.

21. The method according to claim 18 wherein said subject is a mammal selected from the group consisting of humans, horses, dogs and cats.

22. The method according to claim 18 wherein said treatment is due to a gingival injury [symbol]
gingiva, gingival atrophy or shrinkage, acute or chronic wounds/erosions or a scar.

24. The method according to claim 18 wherein said gingival injury is a traumatic wound incurred by surgery, a burn, radiation, or accident.

25. A composition comprising isolated NBDS cell culture supernatant.

26. The composition according to claim 25 wherein said composition further comprises one or more stem cell factors, wnt-factors, growth factors, cytokines or chemokines.

27. The composition according to any one of claim 25 or 26 further comprising a polymer.

28. The composition according to claim 27 wherein said polymer is selected from the group consisting of hyaluronic acid, collagen, polyethylene glycols, poly-lactic acids, and poly caprolactones.

29. The composition according to any of claim 25 or 26 further comprising an extracellular matrix.

30. The composition according to any one of claim 25 or 26 further comprising a non-biodegradable, or, biodegradable scaffold.

31. The composition according to any one of claim 25 or 26 further comprising a paste, paste, cream, gel, or emulsion.

32. The composition according to any one of claim 25 or 26, for use as a coating on a medical device.

33. A medical device, comprising a composition according to claim 32, and a medical device.

34. The medical device according to claim 33 wherein said medical device is a dental implant.
